(19) United States
(12) Patent Application Publication
(10) Pub. No.: US 2012/0128754 A1
(43) Pub. Date: May 24, 2012

(54) TREATMENT OF EYE DISCOMFORT BY TOPICAL ADMINISTRATION OF A COOLING AGENT TO THE EXTERNAL SURFACE OF THE EYELID

(76) Inventor: Edward Tak Wei, Berkeley, CA (US)

(21) Appl. No.: 13/261,061

(22) PCT Filed: Jun. 4, 2010
(86) PCT No.: PCT/GB2010/001094

§ 371 Date: Feb. 9, 2012

Related U.S. Application Data
(60) Provisional application No. 61/217,834, filed on Jun. 5, 2009.
(60) Provisional application No. 61/270,214, filed on Jul. 6, 2009.

(51) Int. Cl. A61K 31/215; A61P 27/02; A61K 31/728; A61K 9/70; A61K 31/717

(52) U.S. Cl. 424/443; 514/529; 514/57; 514/54

(57) ABSTRACT

The present invention pertains generally to the field of ocular treatment, and more specifically to the use of a liquid cooling agent composition comprising a cooling agent for the treatment of (e.g., the alleviation of symptoms of; the amelioration of) eye discomfort. The preferred cooling agent is (1R,2S,5R)-2-isopropyl-5-methyl-cyclohexanecarboxylic acid 2,3-dihydroxy-propyl ester (referred to herein as CPS-030). The liquid cooling agent composition is topically administered to at least a portion of the external surface of the eyelid (preferably the closed eyelid) of the eye to be treated. Preferably, the liquid cooling agent composition is carried on or in a wipe, pad, or towelette, for example, an eye wipe.

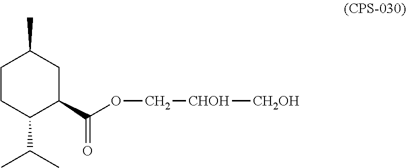

### RELATED APPLICATIONS

This application is related to U.S. provisional patent application No. 61/217,834 filed 5 Jun. 2009 and U.S. provisional patent application No. 61/270,214 filed 6 Jul. 2009, the contents of both of which are incorporated herein by reference in their entirety.

### TECHNICAL FIELD

The present invention pertains generally to the field of ocular treatment, and more specifically to the use of a liquid cooling agent composition comprising a cooling agent for the treatment of (e.g., the alleviation of symptoms of; the amelioration of) eye discomfort. The preferred cooling agent is (1R,2S,5R)-2-isopropyl-5-methyl-cyclohexanecarboxylic acid 2,3-dihydroxy-propyl ester (referred to herein as CPS-030). The liquid cooling agent composition is topically administered to at least a portion of the external surface of the eyelid (preferably the closed eyelid) of the eye to be treated. Preferably, the liquid cooling agent composition is carried on or in a wipe, pad, or towelette, for example, an eye wipe.

### BACKGROUND

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “an adjunctive ocular drug” includes mixtures of two or more such drugs, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

The eye surfaces are exposed to the external environment. These anatomical structures—eyelids, front (anterior) part of the eyeball, conjunctiva, lachrymal system, precorneal film and cornea—are subject to injury by physical, chemical and biological agents. The results of injury to the eye surfaces are symptoms of discomfort, defined as blurring of vision, itching, irritation, fatigue in vision, a sense of dryness, burning sensations, and pain. The signs of injury in the eye are redness, swelling, and increased blood flow. Ophthalmic products such as solutions, ointments, and inserts are used to manage the symptoms and signs of eye injury.

The most common form of drug delivery to treat eye disorders is to administer drug solutions in the form of eye drops. This method is preferred to ointments and inserts because of ease and lower costs of preparation, patient familiarity with procedures of drug dosing, and the lower frequency of side effects.

It is recognized, however, that eye drops are a relatively inefficient method of delivery because individual eye drops range from 20 to 45 μL in volume, whereas the precorneal space in normal subjects is about ˜7 μL per eye. The excess volume rolls down the cheek or may be absorbed into the nasolacrimal duct. Eye drops are also difficult to administer because the patient is taught to recline their head at a 45 to 55° angle and to manually coordinate delivery while keeping their eyes open.

Nevertheless, conventional eye drops represent ˜90% of marketed formulations for eye disorders. This can be seen at the pharmacy where eye drop products for dry eyes (e.g., Systane™) and eye irritation (e.g., Visine™) are on display.

It is known that an ice pack applied to the eye will relieve the pain and discomfort of eye injury. Fujishima et al., 1997, showed that cooling will relieve the pain and inflammation in the eye after cataract surgery. In healthy volunteers, discomfort in the eye induced by pressure on the cornea with a nylon microfilament was much better relieved by artificial tears kept at 4° C. than by tears at 25° C. or 36° C. Thus, cooling is recognized as a potential method for relieving sensory discomfort in the eye.

In the patent literature, chemical cooling agents such menthol (in combination with antihistamines) (see, e.g., Noyori et al., 2000) or p-menthane carboxamides (see, e.g., Bahram et al., 2005) have been proposed for use in eye drops to relieve sensory discomfort. Rohto Pharmaceuticals sells in the United States eye drops labeled as “Cool”, “Ice”, and “Arctic” to relieve redness and to soothe eye strain. In two of these formulations, the cooling ingredient is menthol, which is listed as an “inactive ingredient.” Menthol is an irritant and, in the inventor's experience with the “Ice” and “Arctic” formulations, they cause a strong initial stinging sensation in the eye, the intensity of cooling is limited, and the duration of cooling is less than 10 minutes.

Eye refreshment in a normal subject is sometimes desirable to give a “bright-eyed” alert appearance and to improve visual acuity. Thus, the Rohto-type of eye products fall in the cosmeceutical category. Eye fatigue and eye irritation is also a common symptom of many disorders. One important condition is known as “dry eyes”, which is caused by decreased tear formation and exacerbated, for example, by a dry climate, air pollutants, an increased use of contact lenses, excessive staring at computer screens, and ageing. The estimated prevalence of dry eyes in the United States is about 10 to 30% of the population over age 40, with about 4.9 million severe cases requiring specific medical treatment. Consequently, there is a need for improve treatments of eye discomfort.

### SUMMARY OF THE INVENTION

One aspect of the invention pertains to a method of treating eye discomfort in a human subject, comprising topical administration to at least a portion of the external surface of the eyelid of the eye to be treated, of a therapeutically-effective amount of a liquid cooling agent composition which comprises a cooling agent.

In one embodiment, the method is a method of treating eye discomfort in a human subject, comprising topical administration to at least a portion of the external surface of the closed eyelid of the eye to be treated, of a therapeutically-effective amount of a liquid cooling agent composition which comprises a cooling agent.

Another aspect of the present invention is a cooling agent for the treatment of eye discomfort in a human subject by topical administration of a liquid cooling agent composition comprising said cooling agent to at least a portion of the external surface of the eyelid of the eye to be treated.

In one embodiment, the cooling agent is a cooling agent for the treatment of eye discomfort in a human subject by topical administration of a liquid cooling agent composition comprising said cooling agent to at least a portion of the external surface of the closed eyelid of the eye to be treated.

Another aspect of the present invention is use of a cooling agent in the manufacture of a medicament, wherein said medicament is a liquid cooling agent composition comprising said cooling agent, for the treatment of eye discomfort in a human subject by topical administration of said liquid cooling agent composition to at least a portion of the external surface of the eyelid of the eye to be treated.

In one embodiment, the use is use of a cooling agent in the manufacture of a medicament, wherein said medicament is a liquid cooling agent composition comprising said cooling agent, for the treatment of eye discomfort in a human subject by topical administration of said liquid cooling agent composition to at least a portion of the external surface of the closed eyelid of the eye to be treated.

Another aspect of the present invention is a wipe, pad, or towelette carrying a liquid cooling agent composition comprising a cooling agent, wherein the cooling agent is a polyhydroxyalkyl ester of (1R,2S,5R)-2-isopropyl-5-methyl-cyclohexanecarboxylic acid.

In one embodiment, the wipe, pad, or towelette is a wipe, pad, or towelette carrying a therapeutically-effective amount of a liquid cooling agent composition comprising a cooling agent, wherein the cooling agent is a polyhydroxyalkyl ester of (1R,2S,5R)-2-isopropyl-5-methyl-cyclohexanecarboxylic acid.

In one embodiment, the cooling agent is a polyhydroxyalkyl ester of (1R,2S,5R)-2-isopropyl-5-methyl-cyclohexanecarboxylic acid.

In one embodiment, the cooling agent is (1R,2S,5R)-2-isopropyl-5-methyl-cyclohexanecarboxylic acid 2,3-dihydroxy-propyl ester.

In one embodiment, the cooling agent is present in the liquid cooling agent composition at a concentration of 0.01 to 1% w/v.

In one embodiment, the cooling agent is present in the liquid cooling agent composition at a concentration of 0.1 to 1% w/v.

In one embodiment, the liquid cooling agent composition is water further comprising the cooling agent or a saline solution further comprising the cooling agent.

In one embodiment, the liquid cooling agent composition is water further comprising the cooling agent.

In one embodiment, the liquid cooling agent composition is an isotonic saline solution further comprising the cooling agent.

In one embodiment, the liquid cooling agent composition further comprises an adjunctive ocular drug.

In one embodiment, the adjunctive ocular drug is a polymer lubricant, hypromellose, polyethylene glycol 400, hyaluronan, or propanediol.

In one embodiment, the liquid cooling agent composition is carried on or in a wipe, pad, or towelette.

In one embodiment, the liquid cooling agent composition is carried in an absorbent wipe, pad, or towelette.

In one embodiment, the topical administration is by wiping said wipe, pad, or towelette over the closed eyelid.

In one embodiment, the topical administration is by wiping said wipe, pad, or towelette over the closed eyelid from the medial to the lateral canthus.

In one embodiment, the eye discomfort is eye discomfort caused by extended wear of contact lenses, by eye strain and fatigue, by air pollutants, or by excessive exposure to the sun.

In one embodiment, the eye discomfort is eye discomfort caused by conjunctivitis.

In one embodiment, the eye discomfort is eye discomfort caused by dry eye syndrome.

For the avoidance of doubt, the term “medicament” is intended to include, for example, a cosmeceutical, which may be used, for example, to brighten or enliven otherwise tired or fatigued eyes.

As will be appreciated by one of skill in the art, features and preferred embodiments of one aspect of the invention will also pertain to other aspects of the invention.

### BRIEF DESCRIPTION OF THE DRAWINGS

FIG. 1 is a graph of cooling intensity versus hours after application of CPS-030 with eye wipes for four dosages (128 μg, 64 μg, 32 μg, and 16 μg; per eye), and illustrates the dose-response data for CPS-030 on cooling sensations after application with eye wipes.

### DETAILED DESCRIPTION

The inventor has made the surprising and unexpected discovery that a cooling agent applied to the closed eyelids with a wipe, pad, or towelette provides long-lasting relief of eye discomfort without eye irritation.

For example, a 2 inch×2 inch (5 cm×5 cm) sterile pad used as a substrate to deliver the cooling agent CPS-030, which is provided at a concentration of 4.0 mg/mL of saline at a volume of 16 μL per eye, refreshes the eyes for approximately 1 hour.

This administration of a cooling agent for relief of eye discomfort has not been previously described.

The inventor has also made the surprising and unexpected discovery that certain cooling agents also evoke a sense of wetness, accompanied by an improvement in visual acuity.

Eye drops are the traditional method for delivering drugs to the ocular surface but this method is inefficient and does not work well for cooling agents because sensory discomfort is evoked from the corneal surface by the bolus delivery of the cooling agent.

By contrast, eye wipes moistened with an evenly dispersed liquid composition of a cooling agent, such as CPS-030, can be used to selectively deliver the active ingredient to receptors in the skin above the eyes and on the edges of the eyelids (with instruction to the subject to keep the eyes closed during application), and do not evoke eye irritation. The delivered cooling agent is able to evoke sensations of refreshment and soothing and to relieve eye discomfort.

Thus, a wipe, pad, or towelette (e.g., an eye wipe) carrying, for example, the preferred cooling agent CPS-030, provides a substantial therapeutic improvement over eye drops.

### Neurophysiological Mechanisms of Drug Action:

It may be asked: why is a wipe on the closed eyelids effective for relief of eye discomfort but eye drops are not?

The skin of the eyelids is composed of stratified, keratinized epithelial cells. At the eyelid margin, the cells transit to mucocutaneous tissue which is composed of non-keratinized epithelial cells. The surface of the eye—the conjunctiva, the sclera, and the cornea—has five to six layers of epithelial cells which are densely innervated with sensory nerve endings. Delivery of the cooling agent to the eye surface has to be minimized to avoid eye irritation, and this can be achieved with an eye wipe.

The eyelids, conjunctiva, and cornea contain sensory nerve endings that respond to stimuli that range from touch and pressure, cooling, warmth, and pain. The sensory receptors are located in branches of the trigeminal nerve: (a) the supraorbital nerve (a branch of the frontal nerve, which in turn is a division of the ophthalmic nerve, the smallest branch of the trigeminal nerve) innervates the upper eyelids and the conjunctiva, (b) the infraorbital nerve innervates the lower eyelids, and (c) the long ciliary nerves (a branch of the nasociliary nerve, which in turn is a division of the ophthalmic nerve, the smallest branch of the trigeminal nerve) innervates the cornea. These sensory afferents then project rostrally to the brainstem dorsolateral nucleus where afferent nerve impulses are interpreted as sensations from the ocular surface.

The types of sensory input that evoke afferent nerve discharge from the cornea have been characterized by studies of single unit recordings from the ciliary nerve (see, e.g., Belmonte et al., 2004). About 15% of corneal fibers, all fast conducting A-type, respond exclusively to mechanical force. About 70% of corneal fibers, mostly slow-conducting C-fibers, respond only to noxious stimuli (e.g., heat above 39° C., cold below 29° C., and chemical irritants) and are called polymodal nociceptors. A third category of corneal nerve fibers, about 10-15%, are A_{δ} and C fibers that discharge spontaneously at rest and increase their firing rate when the normal temperature of the corneal surface (˜33° C.) decreases by ˜0.1° C. These are the cold-sensitive receptors that respond to “innocuous” cooling, that is, in the temperature ranges above 29° C. and below 39° C.

The corneal surface is densely packed with polymodal nociceptors and is exceptionally sensitive to painful stimuli. The skin of the eyelids, by contrast, can detect coolness and cold without pain and does not have the exquisite sensitivity of the naked nerve endings on the cornea.

The molecules associated with cold detection on the nerve endings are thought to be ion channel proteins. TrpM8, a non-selective ion channel of the Trp-M (melastatin) family is an important candidate for detection of coolness, but other receptors, such as potassium channels (Trek-1, Trek-2 and Traak), and sodium channels (EnaC), and TrpA1, have also been implicated (see, e.g., Belmonte et al., 2009).

The inventor has discovered that, by creating an apparatus or a method of delivery of a cooling agent to the skin of the eyelids, cooling and pain relief can be achieved without activation of the afferents linked to the long ciliary nerves afferents of the cornea.

And so, by wiping a liquid composition containing a cooling agent onto the skin of the eyelids (from the medial to the lateral canthus, or vice versa) with the eyes closed, contact of the cooling agent with the cornea is avoided or minimized, and the active ingredient is delivered to receptors on the eyelids. The innocuous cooling sensation that results refresh, awaken, and counteract eye discomfort and can be achieved without irritation or pain.

The neuronal pathways by which cool signals may counteract or interdict signals of irritation and discomfort in the brain are explained in, for example, in Wei, 2005 (see especially paragraph [025] therein). The “gating” or sensory over-ride of nociceptive inputs provides the basis by which cooling brings anti-nociceptive or analgesic relief.

An additional surprising and unexpected effect obtained with the application of the preferred cooling agent, CPS-030, to the closed eyelids was an increased sensation of “wetness”. This effect was seen without increased tear production or irritation and may have special benefit for relieving a sense of dryness on the eye surface. A second unusual effect seen, as described in the examples below, is an improvement in visual acuity. It is possible that eye strain can be relieved by cooling and allows better focus and perception. Both of these effects (an increased sensation of wetness and an improvement in visual acuity) have not been described in the scientific literature as a consequence of application of cooling agents to the closed eyelids.

### Drug Delivery Considerations:

Ophthalmic solutions, administered onto the eye surface in the form of eye drops, are the most common form of drug delivery to treat anterior eye disorders. This method is preferred to ointments and inserts because of ease and lower costs of preparation, patient familiarity with procedures of drug dosing, and the lower frequency of side effects.

It is recognized, however, that eye drops are a relatively inefficient method of delivery. Individual eye drops range from 20 to 45 μL per drop, whereas the precorneal space in normal subjects average ˜7 μL per eye. Thus, the pulsatile delivery of an eye drop bolus strikes the curved surface of the eye but there is little time for the active ingredient to reach target receptors. The excess volume splashes on the eye surface, rolls down the cheek, or may be absorbed into the nasolacrimal duct. The contact time of the eye drop with the ocular surface is less than 1 minute and washout is further accelerated by the blink reflex and tear turnover. Eye drops are also difficult to administer because the patient is taught to recline their head at a 45 to 55° angle and to manually coordinate delivery while keeping the eyes open. Nevertheless, conventional eye drops represent ˜90% of marketed formulations for eye disorders. This can be seen at the pharmacy where eye drop products for dry eyes (e.g., Systane™) and eye irritation (e.g., Visine™) are prominently on display.

Drug delivery to eye targets must meet rigorous standards. As defined in Food and Drug Administration regulations (see 21 CFR Chapter 1: 349.3, 4-1-01 Edition: Ophthalmic drug products for the over-the-counter human use), an ophthalmic drug product is sterile (in manufacture) and is a product to be applied to the eyelid or instilled in the eye. Certain defined concentrations of buffering agents, demulcents, emollients, and vasoconstrictors are allowed in ophthalmic products. Ideally, the formulated product is free of preservatives, easy to use, sterile, has a neutral pH and isotonicity, delivers a reliable single dose onto the target, causes minimal discomfort, and does not diminish visual acuity.

A surprising discovery here is that these criteria can be met (in the relief of eye discomfort with a cooling agent), for example, by careful selection of an active ingredient, dissolving the active ingredient in water or an isotonic neutral saline solution, filtering the solution to remove microbial organisms, and delivering the sterile liquid composition via a single-dose unit eye wipe.

### Design of Eye Wipes:

Pre-moistened wipes, pads, or towelettes are used in personal care products, for example, to wipe one's hands, to wipe a baby's skin after a diaper change, or to remove make-up on the face and around the eyes (e.g., Pond's 6 inch×8 inch (15 cm×20 cm) Clean Sweep Cleansing and Make-up Remover Towelettes). The design of wipes is well known to those of skill in the art and generally each is packaged as a single-use sealed unit or in a multi-unit dispenser. For single units, suitable wrapper materials are those which are relatively vapor impermeable, to prevent drying out of the wipe, and able to form a “peelable” seal. Examples of suitable eye wipe/pad materials for practicing the invention include a polyamide (20% nylon)-polyester, a rayon (70%)-polyester (30%) formed fabric, a polypropylene nonwoven fabric, polyethylene terephthalate (PET), polyester polypropylene blends, and microfibers (synthetic or natural fibers that measure less than one denier or one decitex). An example of a wipe packaging is Walgreens Lens Cleaning Wipe which can be purchased at 120 units per box. A suitable size for an eye wipe is exemplified by Walgreens Sterile Pad, 2 inch×2 inch (5 cm×5 cm) (made of rayon-polyester), which can be used to deliver the active ingredient to the eye surface. Using these pads as a substrate, and based on two wipes per eye, the liquid solution delivered per eye is estimated to be ˜16 μL.

It is not necessary that the wipes be individually packaged; they may also be packaged in suitable multi-unit containers, for example, so that the cooling agent solution does not evaporate. Alternatively, the cooling agent solution may be added to individual wraps from a reservoir such as a plastic bottle.

Wei, 2008 describes delivery of cooling compounds to the skin using a towelette. The wiping of the towelette across skin results in delivery of dermatologically active ingredient(s), meaning that the skin is substantially medicated. As shown in the examples below, the compounds described in Wei, 2008 are not suitable for incorporation into wipes because of limited solubility in water. An ethanol/water mixture with an ethanol concentration of about 5 wt % is required in order to dissolve enough of these compounds to provide a sufficient degree of cooling to relieve eye discomfort; however, at this concentration the ethanol is irritating to the eye surface.

### Selection of Active Ingredient:

Ideally, an active pharmaceutical ingredient (API) in a wipe for the eyelids should be stable, non-toxic, and sufficiently long-acting and potent to counteract eye irritation in a clinically significant indication. The API should be evenly dispersible in a liquid composition into or onto a wipe so that during manufacture the wipe can be moistened with a constant and uniform solution when produced under sterile conditions. For purposes of formulation, the API should preferably be a liquid at standard conditions of temperature and pressure (STP) and soluble in aqueous solutions at neutral pH and/or isotonicity. Sterility of the solution can be achieved by filtration through micropore filters and aqueous solubility will facilitate meeting requirements of sterility, a unit dose dispenser, uniform dose delivery, and formulations free of preservatives.

Following experimentation, the inventor found several compounds that met the requirements of an ideal API. These compounds are p-menthane polyhydroxyalkyl carboxylic acid esters and have the structure shown in Formula 1.

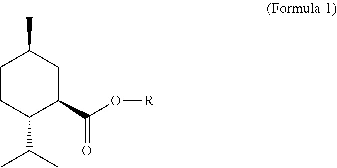

In one embodiment, R is a polyhydroxyalkyl group.

In one embodiment, R is a dihydroxy-C₂₋₄alkyl group.

Preferably, the compound of Formula 1 is liquid at room temperature, readily dispersed into a delivery apparatus, and effective at water soluble concentrations of 0.01 to 1% w/v and effective at less than 1 mg per dose.

Compound CPS-30 is the most preferred compound in this series and is effective, for example, at ˜32 μg/eye delivered via a 2.0 mg/mL solution incorporated into an eye wipe.

CPS-30, shown below, is also known as (1R,2S,5R)-2-isopropyl-5-methyl-cyclohexanecarboxylic acid 2,3-dihydroxy-propyl ester, and corresponds to Formula 1 where R is —CH₂CH(OH)CH₂OH).

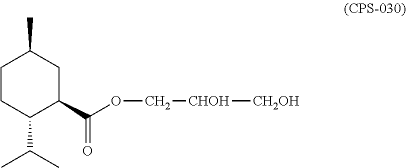

It is noted that the R group may contain one or more chiral centres. For example, in CPS-030, the R group has one chiral centre, marked as C in the following formula: —CH₂CH(OH)CH₂OH. Unless otherwise indicated, the R group may be any stereoisomeric or enantiomeric configuration. For example, for CPS-030, the R group may have the chiral centre in the (R) configuration or the (S) configuration, and CPS-030 may be provided in a form wherein the R group is substantially (R) (e.g., greater than 90% (R) on a molar basis), substantially (S) (e.g., greater than 90% (S) on a molar basis), or a mixture thereof, for example, an equimolar mixture thereof.

Candidate compounds examined that are representative of this group is shown in the Table 1 below. Some of these compounds were described in Watson et al., 1977. None of these compounds have been commercialized.

Some potent cooling agents, which are not liquids at STP, may have sufficient solubility at ˜1 mg/mL in aqueous solutions also to be useful in wipes. These compounds may be compounds of Formula 1, wherein —C(═O)OR contains at least three oxygen atoms in the carbonyl, ether, or hydroxy functional groups.

### Therapeutic Indications:

Eye disorders that cause eye discomfort and are contemplated for treatment by the compositions and methods disclosed herein, preferably by topical application of CPS-030 to closed eyelids by eye wipes, include, but are not limited to:
General eye discomfort: for example, caused by extended wear of contact lenses, by eye strain and fatigue, by air pollutants, or by excessive exposure to the sun.
Conjunctivitis: an inflammation of the conjunctiva that is most commonly caused by allergens, smoke, and pollutants, but which may also be caused by bacterial and viral infection, or by physical agents such as trauma, wind and sunlight.
Dry eye syndrome (keratoconjunctivitis sicca): the inadequate wetting of the ocular surface caused, for example, by inadequate tear secretion or rapid evaporation of tears because of poor tear quality.

The eye wipe with a single cooling agent as the API may be used as a stand alone analgesic wipe. Alternatively, the API may be combined with other agents in the liquid composition in the wipe in order to improve therapy. Examples of such adjunctive ocular drugs are demulcents such as polymer “lubricants”, hypromellose, polyethylene glycol 400, hyaluronan, and propanediol(s). The lubricants increase the elastoviscous properties of the ocular fluids (usually this can be achieved with ophthalmic solutions in the range of 25 to 50 centipoises) and are especially useful for the dry eyes syndrome.

The liquid cooling agent composition may be, for example, water (e.g., purified water) further comprising the cooling agent or a saline solution further comprising the cooling agent. For example, the saline solution may be isotonic (also referred to as normal saline, or physiological saline) of about 0.91% w/v NaCl.

The liquid cooling agent composition may additional comprise suitable buffering agents, for example, boric acid/sodium borate.

The liquid cooling agent composition may additional comprise suitable preservatives, for example, sorbic acid, edetate sodium, and thimerosal (though the latter is less preferred).

The term “treatment,” as used herein in the context of treating a disorder (e.g., eye discomfort), pertains generally to treatment and therapy in which some desired therapeutic effect is achieved, for example, alleviation of symptoms of the disorder, amelioration of the disorder, the inhibition of the progress of the disorder, the reduction in the rate of progress of the disorder, a halt in the rate of progress of the disorder, and cure of the disorder. Treatment as a prophylactic measure (i.e., prophylaxis) is also included. For example, use with patients who have not yet developed the disorder, but who are at risk of developing the disorder, is encompassed by the term “treatment.” For example, treatment of eye discomfort includes the alleviating the symptoms of eye discomfort (e.g., reducing eye discomfort), etc.

The term “therapeutically-effective amount,” as used herein, pertains to that amount of cooling agent or liquid cooling agent composition which is effective for producing some desired therapeutic effect, commensurate with a reasonable benefit/risk ratio, when administered in accordance with a desired treatment regimen.

Why Eye Wipes are Better than Eye Drops for the Delivery of Cooling Agents:

For equivalent concentrations of cooling agents applied to the eyes, eye wipes are effective, but eye drops are not, because eye drops produce adverse sensations such as stinging, irritation and pain. This phenomenon is explained as:
(a) Eye drops are delivered to the naked eyeball and, because of the positioning of the eye dropper, provide a pulse entry of drug onto the corneal surface which is densely innervated by sensory fibers that convey the sensations of pain and irritation. Any cooling agent delivered as an eye drop is thus likely to evoke unpleasant sensations by impacting on nerve endings on the corneal surface.
(b) When an eye wipe is used, drug delivery is applied to the closed eyelids, and the application is onto the keratinized skin covering the eyeball. Some of the solution will contact the eyelid margins, and, to a smaller extent, the conjunctiva, but the corneal surface is avoided. Sensory nerve endings associated with coolness are known to be present in the skin, the eyelids, and the conjunctiva and it is the activation of these nerve endings that provide the sensations of refreshing coolness and cold.
(c) Quantitative measures illustrate the difference between eye drops and wipes. If an average eye drop is 35 μL and two drops are applied per eye, the total is 70 μL. Most of this volume is not delivered because the precorneal space in normal subjects is about ˜7 μL. By contrast, each wipe delivers ˜16 μL per eye, and the delivery is more localized to the eyelids and conjunctiva so that the desired pharmacological effect is obtained.
(d) It should be noted that conventional therapy does not recognize the use of eye wipes to deliver pharmacologically active substances to the ocular surface. A recent review of drug delivery to the eye (see, e.g., Novack, 2009) does not mention this mode of drug transfer.

In view of the above considerations, an eye wipe, by comparison to an eye drop, avoids an abrupt bolus dose to the cornea, is more efficient in delivery of the cooling agent to its relevant target receptors, and the duration of action is longer because of increased contact with the skin above the eyeballs and with the edges of the eyelids. Hence, an eye wipe with a cooling agent will enable the desired response of a soothing and refreshing feeling in the eye without irritation and sting.

### Kits

One aspect of the invention pertains to a kit comprising (a) a liquid cooling agent composition which comprises a cooling agent, as described herein, e.g., preferably provided in a suitable container and/or with suitable packaging; and (b) instructions for use, e.g., written instructions on how to administer the liquid cooling agent composition.

The written instructions may also include a list of indications for which the active ingredient is a suitable treatment.

The subject/patient may be, e.g., a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human. In a preferred embodiment, the subject/patient is a human.

### EXAMPLES

The following examples are provided solely to illustrate the present invention and are not intended to limit the scope of the invention, as described herein.

### Example 1

The general procedure for testing was to weigh an active ingredient and to disperse it within a saline solution (i.e., Bausch & Lomb, Sensitive Eyes Plus Saline Solution) at stock concentrations of 10 to 20 mg/mL. Test solutions were further diluted with isotonic saline. For single assays, a 1 mL scaled pipette was used to dispense 0.8 to 1 mL of test solution onto a sterile 2″×2″ (5 cm×5 cm) pad, made of rayon-polyester formed fabric (Walgreens Sterile Gauze Pads) weighing on average 285±9 mg.

For safety precautions, the wet pad was first tested by applying the pad to the philtrum, to determine if there was any irritation or pain. No irritancy was observed with any of the test substances.

In the next trials, the wet pad was wiped twice over the closed eyelids of each eye from the medial to the lateral canthus. By subtraction, the loss of weight of the pad averaged 32±1 mg. Thus, each eye received ˜16 μL of the test solution per eye. For a 1 mg/mL solution this was equivalent to ˜16 μg per eye.

The presence of cooling sensations was recorded at 2 and 5 minutes after wiping and at 5 minute intervals thereafter. The intensity of the subjective eye sensation was rated as 0, 1, 2 or 3 with: 0 as no change; 1 as slight coolness or cold; 2 as clear-cut signal of coolness or cold; and 3 as comfortable and soothing sensations of cooling or cold. Recording was continued until two successive zeroes were obtained. The data were plotted using SigmaPlot™ (Systat Software, Point Richmond Calif.).

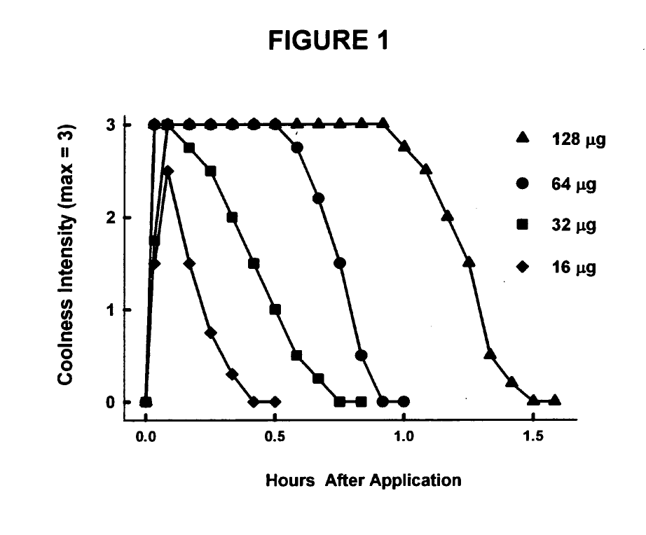

The results for compound CPS-030 are illustrated in FIG. 1, and reflect the averaged values of 6 to 8 separate trials in the same individual.

FIG. 1 is a graph of cooling intensity versus hours after application of CPS-030 with eye wipes for four dosages (128 μg, 64 μg, 32 μg, and 16 μg; per eye), and illustrates the dose-response data for CPS-030 on cooling sensations after application with eye wipes. The dose reported in μg is per eye, based on solutions of 1.0, 2.0, 4.0 and 8.0 mg of CPS-030 per mL of saline, applied in a volume of 16 μL per eye. At the 32 μg, 64 μg and 128 μg dose, cooling was accompanied by a sensation of “wetness” and increased visual acuity. At all dosages tested, there were no sensations of sting, irritation, or pain. Confirmatory trials of cooling action for CPS-030 were obtained in 4 individuals.

### Example 2

(R)-2-[((1R,2S,5R)-2-isopropyl-5-methyl-cyclohexanecarbonyl)-amino]-propionic acid ethyl ester, referred to as CPS-369 (see, e.g., Wei, 2008) was synthesized and tested. This compound, which is a white solid at room temperature, was dispersed in saline solution by sonication and tested at 1 mg/mL and 2 mg/mL. Robust and refreshing cooling was obtained lasting approximately 1.5 and 2.5 hours, respectively. It was noted, however, that after testing, if the subject was to get their eyelids wet, for example, from perspiration, taking a shower, or washing the face with a towel, the cooling sensations returned, sometimes with an intensity that could cause discomfort. This side-effect, which was attributed to activation of residual solid test substance deposited on the eyelid which then washed onto the corneal surface, indicated that solid cooling agents were not ideal as active ingredients. An active ingredient should be water soluble so that it can be flushed out of the eye after application and not leave a residue. Water insoluble cooling agents are not ideal for practicing the invention.

To solubilize CPS-369, it was dissolved 30 mg in 0.1 mL of absolute ethanol and 7.4 mL of glycerol was added, followed by 7.5 mL of distilled water. The CPS-369 remained in solution and was tested on the eyelids. Mild and persistent cooling was observed. Thus, CPS-369 was incorporated into a liquid composition that could be added to an eye wipe. However, as glycerol is not a common vehicle for eye solutions, CPS-369 was not considered to be an optimal cooling agent for practicing the invention.

### Example 3

2-[((1R,2S,5R)-2-isopropyl-5-methyl-cyclohexanecarbonyl)-amino]-acetic acid 3-hydroxypropyl ester, referred to as CPS-160 (see, e.g., Wei, 2008) was synthesized and tested. CPS-160 was first computationally predicted to be a water soluble liquid at room temperature. This compound, when synthesized, is a colorless liquid and was solubilized in saline solution and tested at 2 mg/mL. Robust and refreshing cooling was obtained in the orbit lasting approximately 0.6 hours. It was noted, however, that solutions were not stable and became inactive after 2 days when stored at room temperature. Thus, CPS-160 was not considered to be an optimal cooling agent for practicing the invention.

### Example 4

(1R,2S,5R)-2-Isopropyl-5-methyl-cyclohexanecarboxylic acid 2,3-dihydroxy-propyl ester, referred to as CPS-030 (and also known as WS-30) (see, e.g., Watson et al., 1977) was synthesized by esterifying (1R,2S,5R)-2-isopropyl-5-methyl-cyclohexanecarbonyl chloride with 1,2-isopropylidene glycerol, followed by hydrolysis with trifluoracetic acid. The product was a mixture of (1R,2S,5R)-2-isopropyl-5-methyl-cyclohexanecarboxylic acid (R or S)-2,3-dihydroxy-propyl ester. No attempt was made to separate and bioassay the two enantiomers.

Among a series of (1R,2S,5R)-2-isopropyl-5-methyl-cyclohexanecarboxylic esters, CPS-030 was predicted to have the greatest water solubility and the lowest octanol/water partition coefficient (see Table 1).

TABLE 1
Calculated Octanol/Water Partition Coefficients (P) and Water Solubility

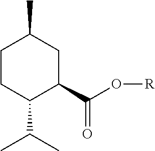

| | | | | Water |
|---|---|---|---|---|
| | | | | solubility |
| No. | Code | R | Log(P) | (mg/mL) |
| 1 | CPS-030 | —CH₂CH(OH)CH₂OH | 1.98 | 26.1 |
| 2 | CPS-065 | —CH₂CH₂OCH₂CH₂OH | 2.46 | 1.1 |
| 3 | CPS-004 | —CH₂CH₂OH | 2.59 | 1.1 |
| 4 | WS-1 | —H | 3.0 | 0.2 |
| 5 | | —CH₂CH₂CH₂OH | 2.80 | 0.1 |
| 6 | | —CH₂CH₂CH₂CH₂OH | 3.25 | 0.04 |
| 7 | | —CH₂CH(OH)CH₃ | 2.92 | 0.8 |
| 8 | | —CH(CH₃)CH₂OH | 3.03 | 0.8 |
| 9 | | -D-Ala-OMe | 2.8 | 0.1 |
| 10 | WS-31 | -Gly-O-OMe | 2.4 | 0.2 |
| 11 | WS-5 | -Gly-O-Et | 2.9 | 0.06 |
| 12 | CPS-369 | -D-Ala-O-Et | 3.3 | 0.04 |
| 13 | | -D-Ala-O-nPr | 3.8 | 0.01 |
| 14 | | -D-Ala-O-iPr | 3.7 | 0.03 |
| 15 | | menthol | 3.1 | 0.9 |

CPS-030, which is a colorless liquid at room temperature, was miscible and solubilized in saline solution and was tested at 1, 2, 4, and 8 mg/mL. Robust and refreshing cooling was obtained in the orbit. This compound retained biological potency when stored at room temperature for at least three weeks. It has a good safety profile because the expected hydrolysis products are the carboxylic acid and glycerol. The full dose-response analysis of CPS-030 is shown in FIG. 1.

CPS-004 and CPS-065, both liquids at STP, were also tested. CPS-065, at 10 mg/mL, produced mild cooling (<2 coolness units), lasting less than 30 minutes. CPS-004, by contrast, showed a full dose-response range from 32 to 160 μg/eye. Surprisingly, at the higher two doses, CPS-004 produced a brief period of warmth sensations on the eye surface and increased lachrymation. CPS-004 had about half the potency of CPS-030. Neither CPS-004 nor CPS-065 was considered to be an optimal cooling agent for practicing the invention.

It was noted that compounds with more potent cooling activity than CPS-030, e.g., Compounds 9 and 10 in Table 1, can be dissolved in a 1 or 2% ethanol-water solution at 1 mg/mL. These solutions, when applied to the closed eyelids, produced cooling for 15 to 30 minutes, but without any sensations of wetness or increased visual acuity. Higher aqueous concentrations of Compounds 9 and 10 could not be achieved unless the ethanol concentrations were also increased, but this caused irritating eye sensations. Thus, water solubility, as embodied in CPS-030, is a key factor for an ideal API.

### Example 5

A 62-year old male routinely spent about 12 hour each day in front of a computer monitor and developed headaches and blurred vision. A visit to a general physician gave a diagnosis of eye strain, but the addiction to the computer monitor continued. The subject found some relief by putting a cold wet towel on his face, but the relief was only temporary. He volunteered to test an eye wipe containing 0.8 mL of a 4 mg/mL solution of CPS-030 and was given instructions on how to wipe the solution onto the eyelids with the eyes closed. Within two minutes the subject reported cooling and soothing sensations on his eyes that were maintained for at least two hours. During this time, the subject watched a basketball playoff game on television and noted that he could clearly distinguish scores on the 46 inch (117 cm) HDTV monitor which were previously indecipherable. For example, he could clearly see scores of 65 vs. 66, and of 98 vs. 99. This was unexpected, and the subject attributed the increase in visual acuity to a sense of heightened alertness and relaxation which was produced by the cooling sensations in his eyes. The subject continues to use the eye wipes as an aid to his vision on an “as needed basis”, and the improved visual acuity is a reproducible event.

### Example 6

A 80-year old male had a diagnosis of “dry eyes syndrome” of 2 years duration. His principal complaints were of dryness, itch, and discomfort at the corner of his eyes and blurred vision. These symptoms were present on a daily basis at various levels of intensity. He used Systane™ and Restasis® eye drops but said they were minimally effective. It was not clear if he adhered to a strict regimen of self-administration, but he complained of the eye drops running down his cheeks and the expense of the medications. When the symptoms were severe, the subject found some relief by wetting his eyes with cold tap water, but the relief was only temporary. He volunteered to test an eye wipe containing 0.8 mL of a 4 mg/mL solution of CPS-030, and was given instructions on how to apply the solution onto the eyelids with the eyes shut. Within two minutes, the subject reported cooling and soothing sensations on his eyes that were maintained for at least 1 hour. The subject remarked on the sensation of “wetness”, and a feeling of soothing and comfort. The subject used the eye wipes for symptomatic relief on an “as needed basis” for three months and expressed satisfaction at the relief of eye discomfort.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Bahram, A. et al., 2005, “Use of cooling agents to relieve mild ocular irritation and enhance comfort”, US Patent Publication No. 2005/0137166 A1 published 23 Jun. 2005.
Belmonte, C. et al., 2004, “Nerves and sensations from the eye surface”, Ocul. Surf., Vol. 2, pp. 248-253.
Belmonte, C. et al., 2009, “Converting cold into pain”, Exp. Brain Res., Vol. 196, pp. 13-30.
Fujishima et al., 1997, “Effects of artificial tear temperature on corneal sensation and subjective comfort”, Cornea, Vol. 16, pp. 630-634.
Novack, 2009, “Ophthalmic drug delivery: development and regulatory considerations”, Clin. Pharmacol. Therap., Vol. 85, pp. 539-543.
Noyori, S. et al., 2000, “Ophthalmic solution”, U.S. Pat. No. 6,147,081 issued 14 Nov. 2000.
Watson, H. R. et al., 1977, “Substituted p-Menthanes”, U.S. Pat. No. 4,033,994 granted 5 Jul. 1977.
Wei, 2005, “N-Arylsalkyl-carboxamde compositions and methods”, US Patent Publication No. 2005/0187211 published 25 Aug. 2005.
Wei, 2008, “N-alkylcarbonyl-amino acid ester and N-alkylcarbonyl-amino lactone compounds and their use”, US Patent Publication No. 2008/0227857 A1 published 18 Sep. 2008.

1-60. (canceled)

61. A method of treating eye discomfort in a human subject, comprising; topical administration to at least a portion of the external surface of the eyelid of the eye to be treated of a therapeutically-effective amount of a liquid cooling agent composition which comprises a cooling agent, wherein the cooling agent is (1R,2S,5R)-2-isoproply-5-methyl-cyclohexanecarboxylic acid 2,3-dihydroxy-propyl ester.

62. The method as in claim 61 wherein the cooling agent is present in the liquid cooling agent at a concentration of about 0.01 to about 1% w/v.

63. The method as in claim 62 wherein the liquid cooling agent composition is carried on or in a wipe, pad or towelette.

64. The method as in claim 63 wherein the topical administration is by wiping said wipe, pad or towelette over the closed eyelid.

65. The method as in claim 63 wherein the topical administration is by wiping said wipe, pad or towelette over the closed eyelid from the medial to the lateral canthus.

66. The method as in claim 61 wherein the eye discomfort is caused by extended wear of contact lenses, by eye strain and fatigue, by air pollutants, by excessive exposure to the sun, by conjunctivitis or by the dry eyes syndrome.

67. The method as in claim 63 wherein the liquid cooling agent composition further comprises an adjunctive ocular drug.

68. A wipe, pad or towelette carrying a liquid cooling agent composition comprising: a cooling agent, wherein the cooling agent is (1R,2S,5R)-2-isoproply-5-methyl-cyclohexanecarboxylic acid 2,3-dihydroxy-propyl ester.

69. The wipe, pad or towelette as in claim 68 wherein the cooling agent is present in the liquid cooling agent composition at a concentration of about 0.01 to about 1% w/v.

70. The wipe, pad or towelette as in claim 68 wherein the liquid cooling agent composition further comprises an adjunctive ocular drug.

71. The wipe, pad or towelette as in claim 68 wherein the adjunctive ocular drug is a polymer lubricant, hypromellose, polyethylene glycol 400, hyaluronan or propanediol.

72. The wipe, pad or towelette as in claim 68 wherein the liquid cooling agent composition further comprises water or an isotonic saline solution.
